(19) United States
(12) Patent Application Publication
(10) Pub. No.: US 2025/0352685 A1
(43) Pub. Date: Nov. 20, 2025

(54) Sterilization Tray

(71) Applicant: ASP Global Manufacturing GmbH, Schaffhausen (CH)

(72) Inventors: Todd Morrison, Dana Point, CA (US); Doug Truong, Irvine, CA (US)

(73) Assignee: ASP Global Manufacturing GmbH, Schaffhausen (CH)

(21) Appl. No.: 19/213,410

(22) Filed: May 20, 2025

Related U.S. Application Data
(60) Provisional application No. 63/649,501, filed on May 20, 2024.

(51) Int. Cl. A61L 2/26

(52) U.S. Cl. CPC A61L 2/26; A61L 2202/24

(57) ABSTRACT

An aspect of the present disclosure is a tray for containing a medical device during a sterilization process. The tray includes a bottom portion comprising a plurality of perforations extending through a surface of the bottom portion. The tray further includes a plurality of side portions extending from the bottom portion, the plurality of side portions each comprising a plurality of slots extending through a surface of each respective side portion. The tray further includes a cover configured to releasably engage the plurality of side portions, the cover comprising a plurality of openings extending through a surface of the cover. Trays disclosed herein are designed to minimize residual liquid leakage and freezing and maximize diffusion of sterilant through the tray and into contact with the medical device(s).

### CROSS-REFERENCE TO RELATED APPLICATIONS

This application claims the benefit of the filing date of U.S. Provisional Patent Application No. 63/649,501, filed on May 20, 2024, the disclosure of which is hereby incorporated herein by reference.

### BACKGROUND OF THE INVENTION

Medical devices are preferably sterilized before use in order to minimize the likelihood that a device contaminated by, e.g., microorganisms might be used on a subject. Tainted devices and instruments could cause an infection in the subject, leading to potentially extreme adverse outcomes. This applies to devices that are utilized in both less invasive procedures (e.g., colonoscopies) and more invasive procedures (e.g., orthopedic procedures).

Various sterilization techniques have been developed and may employ the use of sterilants including one or a combination of steam, ethylene oxide, chlorine dioxide, ozone, peracetic acid, and hydrogen peroxide. Often these chemical sterilants are employed in a gaseous and/or a plasma form. For these techniques, sterilization is typically conducted within a sterilization chamber of a sterilization system, with certain chemical sterilization techniques, such as those using hydrogen peroxide, making use of a vacuum chamber that is not only capable of achieving low pressures, but of also introducing and withdrawing sterilants therefrom. Some chemical sterilization processes, such as those that use ethylene oxide, require water vapor within the vacuum chamber to be effective. However, for other chemical sterilization processes, such as those that use hydrogen peroxide, water in vapor, liquid, or solid form within the vacuum chamber may decrease effectiveness or cause the process to abort/cancel.

A typical chemical vapor sterilization process for medical devices begins with medical-facility personnel preparing the devices for sterilization by washing the instruments with water and/or a washing solution to remove solids and liquids from the instrument. The personnel then dries the instruments, (e.g., by using heat, medical-grade compressed air, and/or towels) and perhaps wraps them in a wrap suitable for sterilization, which acts as a barrier to microorganisms but that permits passage of a sterilant therethrough. Instruments wrapped in a wrap are sometimes referred to as a sterilization pack or load and can be thermally nonconductive. The load is then placed into the vacuum chamber of the sterilization system and the chamber is closed (scaled), typically by closing the chamber's door. The chamber may be heated, which may help vaporize water that may be within the chamber. Next, the atmosphere in the chamber, which may include the unwanted water vapor, is evacuated. In some sterilization procedures, air within the vacuum chamber may be excited to form an air plasma, which may further aid in vaporizing water for removal from the chamber.

After achieving a low pressure, sometimes referred to as a vacuum or a rough vacuum, a sterilant is introduced into the chamber, either in gaseous or vapor form or as a mist that vaporizes in the low-pressure environment of the chamber. The added gas in the chamber slightly raises the pressure in the chamber. The sterilant spreads throughout the chamber, entering small or confined spaces, such as cracks, crevices, and lumens in the medical devices contained therein. The sterilant bathes the medical devices, which kills bacteria, viruses, and spores disposed upon and within the devices that it contacts. In some sterilization procedures, particularly low-temperature procedures that utilize hydrogen peroxide, the hydrogen peroxide gas may be excited via an electric field to change the gas into a plasma. Finally, the sterilant is evacuated from the chamber and the chamber is returned to the ambient pressure. After the sterilization process has ended, the instruments may be removed from the chamber.

Medical devices or loads are typically positioned in wrapped sterilization trays prior to being placed into the vacuum chamber of the sterilization chamber and system. These sterilization trays must allow residual liquid (e.g., water or alcohol) to be vaporized and removed from the load prior to introducing a sterilant into the chamber. Additionally, these sterilization trays must allow as much access as possible for the sterilant to contact the load during the sterilization process such that the medical device fully bathes within the sterilant.

However, commercially available sterilization trays do not provide an adequate balance between the removal of residual liquid from the load and access for sterilant to contact the medical device during the sterilization process. The design of these sterilization trays includes a plurality of openings extending therethrough to assist with the drying and sterilization process, but not in a manner to sufficiently address either issue. This causes issues and delays in performing the sterilization process, as residual liquid that is not removed from the medical device and sterilization chamber prior to the sterilization process may freeze on a surface of the sterilization tray or the wrap, rather than be vaporized. Additionally, residual liquid may cause the sterilization process to abort and may require the medical device to undergo an additional drying process. These additional steps will increase the overall time required to sterilize the load and the reduce efficiency of the sterilization process.

Therefore, there exists a need for a sterilization tray that adequately assists in removal of residual liquid from medical devices prior to commencing a sterilization process but does not restrict sterilant from contacting the medical device during the sterilization process.

### BRIEF SUMMARY OF THE INVENTION

An aspect of the present disclosure is a tray for containing a medical device during a sterilization process. In one embodiment, the tray comprises a bottom portion comprising a plurality of perforations extending through a surface of the bottom portion; a plurality of side portions extending from the bottom portion, the plurality of side portions each comprising a plurality of slots extending through a surface of each respective side portion; and a cover configured to releasably engage the plurality of side portions, the cover comprising a plurality of openings extending through a surface of the cover, wherein each perforation of the plurality of perforations comprises an area that is less than an area for each slot of the plurality of slots and for each opening of the plurality of openings.

In one example of the tray, the tray includes a plurality of perforations, plurality of slots, and plurality of openings are equal to a porous surface area, the porous surface area being less than a total surface area of the tray. The porous surface area is equal to 35% to 45% of the total surface area of the tray, and the porous surface area may specifically equal to 42% of the total surface area of the tray.

In another example of the tray, at least one side portion includes a boss to releasably engaging a boss opening of the cover. In another example of the tray, an inner surface of at least one side portion includes a holder configured to receive a chemical indicator or a component of the medical device. In another example of the tray, the tray is wrapped in a material comprised of at least polypropylene. In another example of the tray, the tray is comprised of at least a metal alloy such as aluminum or stainless steel.

In another embodiment, the tray comprises a bottom portion comprising a plurality of perforations extending through a surface of the bottom portion; a plurality of side portions extending from the bottom portion, the plurality of side portions each comprising a plurality of slots extending through a surface of each respective side portion; and a cover configured to releasably engage the plurality of side portions, the cover comprising a plurality of openings extending through a surface of the cover, wherein the plurality of perforations comprises a first surface area less than a total surface area of the bottom portion, the plurality of slots comprises a second surface area less than a total surface area of the plurality of side portions, the plurality of openings comprises a third surface area less than a total surface area of the cover, and wherein the first surface area relative to the total surface area of the bottom portion is less than the second surface area relative to the total surface area of the plurality of side portions and is less that the third surface area relative to the total surface area of the cover.

In one example of the tray, the first, second, and third surface area are equal to a porous surface area, the porous surface area being less than a total surface area of the tray. The porous surface area is equal to 35% to 45% of the total surface area of the tray, porous surface area may specifically equal to 42% of the total surface area of the tray.

In another example of the tray, at least one side portion includes a boss to releasably engaging a boss opening of the cover. In another example of the tray an inner surface of at least one side portion includes a holder configured to receive a chemical indicator or a component of the medical device. In another example of the tray, the tray is wrapped in a material comprised of at least polypropylene. In another example of the tray, is tray is comprised of at least a metal alloy such as aluminum or stainless steel.

Another aspect of the present disclosure is a method for preparing a medical device for sterilization. The method comprises positioning a sterilization tray containing a medical device within a sterilization chamber; changing a condition of the surface of the sterilization tray, wherein the surface of the sterilization tray is conditioned to evaporate a liquid contacting the sterilization tray into a vapor; and evacuating the vapor from the sterilization chamber, wherein at least 10 milliliters of the liquid is evaporated into the vapor and evacuated from the sterilization chamber.

Yet another aspect of the present disclosure is a tray for containing a medical device during a sterilization process. The tray includes a bottom portion including a planar portion and a tapered portion, each of the planar portion and the tapered portion having a plurality of perforations extending therethrough, a plurality of side portions extending from the bottom portion, the plurality of side portions each including a plurality of slots extending through a surface of each respective side portion, and a cover releasably engaged to at least one of the plurality of side portions, the cover including a plurality of openings extending through a surface of the cover. The perforations are smaller than the slots and the slots are smaller than the openings.

In other embodiments of this aspect, the plurality of perforations, plurality of slots, and plurality of openings may define a porous surface area, the porous surface area being less than 50% of a total outer surface area of the tray. The porous surface area may be equal to 35% to 45% of the total outer surface area of the tray. More specifically, the porous surface area may be equal to 42% of the total outer surface area of the tray. The at least one side portion may include a boss releasably engaging a button opening of the cover. An inner surface of at least one side portion may include a holder configured to receive a chemical indicator or a component of the medical device. The may be wrapped in a polypropylene material. The tray may be constructed of a metal, for instance, aluminum or stainless steel.

Yet another aspect of the present invention is a tray for containing a medical device during a sterilization process including a bottom portion including a plurality of perforations extending through the bottom portion, a plurality of side portions extending from the bottom portion, each of the side portions including a plurality of slots extending through each respective side portion, and a cover including a plurality of openings extending through the cover. The plurality of perforations define a first surface area, the plurality of slots define a second surface area, and the plurality of openings define a third surface area, and the first surface area is less than the second surface area and the second surface area is less that the third surface area.

In other embodiments of this aspect the first, second, and third surface areas may define a porous surface area, the porous surface area being less than 50% of an outer surface area of the tray. The porous surface area may be equal to 35% to 45% of the outer surface area of the tray, for instance, the porous surface area may be equal to 42% of the outer surface area of the tray. The at least one side portion may include a boss to releasably engaging a button of the cover. An inner surface of at least one side portion may include a holder configured to receive a chemical indicator or a component of the medical device. The tray may be wrapped in a polypropylene material. The may be constructed of a metal, for instance, aluminum or stainless steel.

Yet another aspect of the present invention is a method of sterilizing a medical device. The method includes the steps of operating a sterilization device to remove at least 10 milliliters of residual liquid from a sterilization tray including a medical device positioned within a sterilization chamber of the sterilization device and operating the sterilization device to sterilize the medical device. The method may further include the steps of positioning the medical device in the sterilization tray and the sterilization tray within the sterilization chamber.

Another aspect of the present invention is a tray for holding medical devices including a base having a base surface and a base configuration of openings on the base surface, the base configuration of openings being configured to facilitate diffusion of a sterilant through the base surface into the tray and to prevent residual liquid in the tray from freezing on a sterilization wrap surrounding the tray.

In other embodiments of this further aspect, the tray may further include a plurality of sides extending from the base, wherein at least one side includes a wall and a side configuration of openings on the wall, the side configuration of openings being configured to facilitate sterilant communication in and out of the tray and to enable vaporization of residual liquid through the side. The tray may also include a lid configured to releasably engage the plurality of sides, the lid having a lid configuration of openings configured to facilitate sterilant communication in and out of the tray and vaporization of residual liquid through the lid. An open area ratio of the lid may be at least 200% of an open area ratio of the base. The tray may further include an accessories shelf having a security wall, the shelf connected to two sides of the plurality of sides, wherein the accessories shelf is configured to receive a sterilization indicator and retain the sterilization indicator based in part on the security wall during and after a sterilization process. The base configuration of openings, the side configuration of openings, and the lid configuration of openings may collectively be configured to form a tray open area ratio, the tray open area ratio being greater than a minimum open area ratio required to achieve efficacy of a sterilization cycle for gastrointestinal endoscopes. The minimum open area ratio may be 40%. The base configuration of openings may have an open area ratio of less than 15-20% to prevent the residual liquid in the tray from freezing on the sterilization wrap. The base surface may include at least one metal alloy, and the base surface and the base configuration of openings may be configured to (1) facilitate a load conditioning process of a sterilization cycle to remove up to 15 milliliters of residual liquid from the tray or (2) prevent the residual liquid from freezing on the sterilization wrap at pressures below 4.6 Torr in a vacuum chamber of the sterilizer. The base configuration of openings may include a plurality of perforations or a plurality of slots, wherein the perforations or slots collectively form an open area ratio of less than 20%. The base surface may be a top surface, and the base may further include a flat bottom surface configured to contact the sterilization wrap to facilitate diffusion of the sterilant through the base configuration of openings into the tray. The of claim 1, wherein the base surface may have a size configured to accommodate a flexible gastrointestinal endoscope having a bending radius for an insertion tube greater than 125 mm.

Yet another aspect of the present invention is a method for making a sterilization tray. This may includes the steps of forming a base configuration of openings in a thermally conductive base of the sterilization tray, the base configuration of openings being configured to prevent residual liquid in the tray from freezing on a thermally nonconductive wrap covering the tray during a sterilization process, and forming a lid configuration of openings in a lid of the sterilization tray, wherein the lid configuration of openings and the base configuration of openings provide an open area ratio of the lid that is at least twice that of the base.

In other embodiments of this method, the base configuration of openings may include a plurality of perforations or a plurality of slots, wherein the perforations or slots collectively form an open area ratio of less than 15%. The base may include at least one metal alloy, and the base and the base configuration of openings may function in synergy to enable a load conditioning process of a sterilization cycle to remove up to 10 milliliters of residual liquid from the tray.

Yet another embodiment of the present disclosure is a tray for holding medical devices including means for preventing residual liquid in the tray from freezing on a sterilization wrap surrounding the tray during a sterilization process and means for evacuating the residual liquid from the tray. This tray may further include means for facilitating passage of vaporized sterilant or moisture through a base, a side, or a lid of the tray.

### BRIEF DESCRIPTION OF THE DRAWINGS

FIG. 1 is a perspective view of a sterilization tray according to one embodiment of the present disclosure.

FIG. 2A is a bottom view of the sterilization tray of FIG. 1.

FIG. 2B is an enlarged view of the bottom view of FIG. 2A.

FIG. 3A is a side view of the sterilization tray of FIG. 1.

FIG. 3B is an enlarged view of the side view of FIG. 3A.

FIG. 4 is another side view of the sterilization tray of FIG. 1.

FIG. 5 is a top view of the sterilization tray of FIG. 1.

FIG. 6 is another perspective view of the sterilization tray of FIG. 1 with a cover removed.

FIG. 7 is another top view of the sterilization tray of FIG. 1 with a cover removed.

FIG. 8 is a top view of a cover of the sterilization tray of FIG. 1.

FIG. 9 is a bottom view of the cover of FIG. 8.

FIG. 10 is top view of a medical device positioned in the sterilization tray of FIG. 1.

FIG. 11 is a perspective view of another medical device configured to be positioned within the sterilization tray of FIG. 1.

FIG. 12 is an enlarged view of the medical device of FIG. 11.

FIG. 13 is a top view of a material wrapped around the sterilization tray of FIG. 1.

FIGS. 14-16 illustrate alternative configurations for bottom surfaces of trays in accordance with the present disclosure.

FIG. 17 illustrates an offset baffle and tray design according to another embodiment of the present disclosure.

### DETAILED DESCRIPTION

Particular embodiments of the present disclosure will be described herein with reference to the accompanying drawings. Also, as used herein, the terms “substantially,” “generally,” and “about” are intended to mean that slight deviations from absolute are included within the scope of the term so modified.

As discussed more fully below, the designs of the present invention are aimed at optimizing sterilization processes by providing a tray for housing one or more medical devices that minimizes residual liquid leakage and freezing and maximizes diffusion of sterilant through the tray and into contact with the medical device(s). It has been found that smaller bottom holes prevent the residual liquid leakage and freezing and maximization of other openings in the tray provides for maximum diffusion of sterilant. Particular embodiments of these concepts will now be discussed.

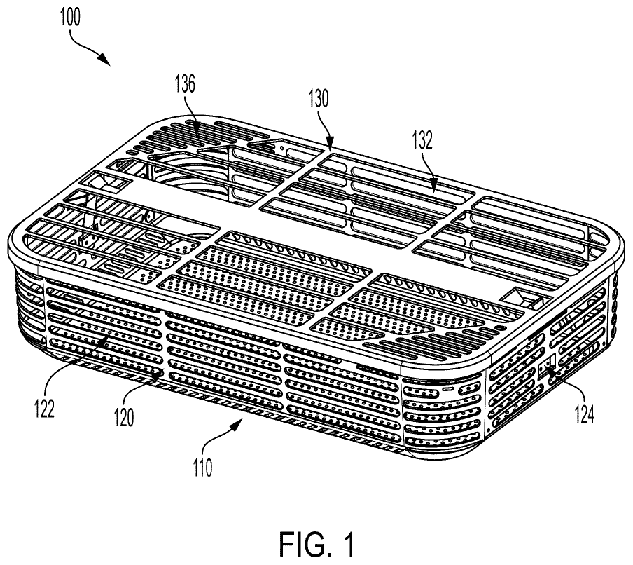

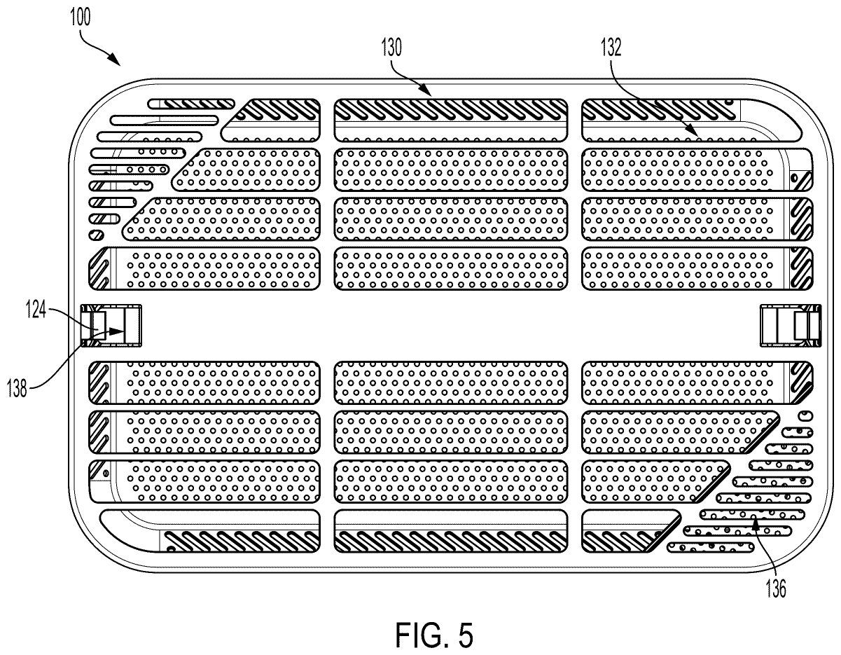

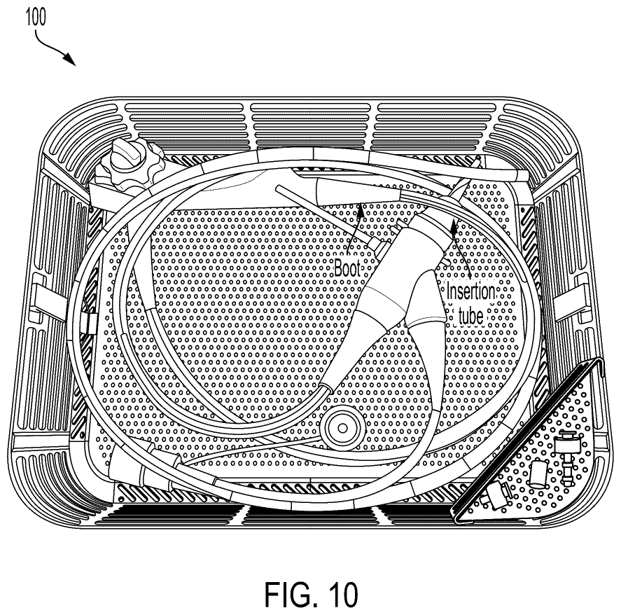

Referring to FIGS. 1-5, a sterilization tray 100 is shown. Sterilization tray 100 includes a bottom surface 110, side surfaces 120 and a cover or top surface 130. These surfaces form a three-dimensional rectangular structure with an essentially open interior. Variations in the overall shape of tray 100 are contemplated and can include, for instance, square, triangular and round three-dimensional structures. Sterilization tray 100 is designed to receive a medical device, as best shown in FIG. 10. The medical device may be an endoscope, such as a gastrointestinal (GI) endoscope, or other type of commonly used medical device that is to be sterilized. Sterilization tray 100 is designed to be used in a sterilization system during a load conditioning and sterilization process, such as any of those shown and described in U.S. Pat. Nos. 9,931,427, 10,799,609, 10,441,672, 11,020,505, 10,458,705, 11,650,011, 10,668, 180, and 11,660,365, the entire disclosures of which are all incorporated by reference herein. Tray 100 can, of course, have other uses including in other sterilization processes such as autoclaving and the like.

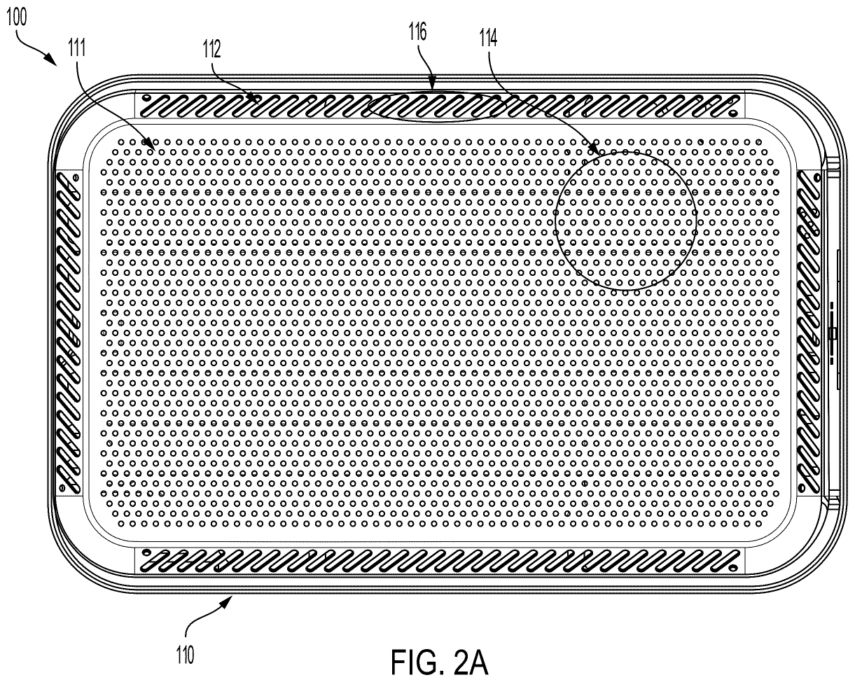

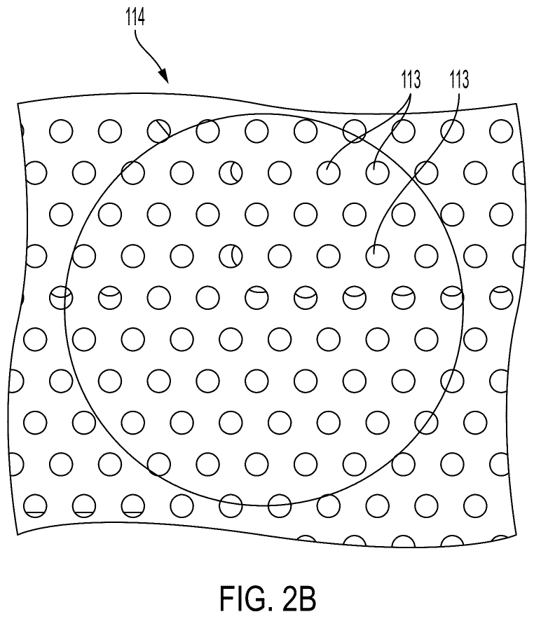

Bottom surface 110 of sterilization tray 100 is best shown in FIG. 2A. Bottom surface 110 includes a generally flat or planar portion 111 with a substantially rectangular shape that is defined by rounded corners. Planar portion 111 of bottom surface 110 includes a plurality of perforations 113 extending therethrough and into an inner space of the sterilization tray 100. Perforations 113 are shown in more detail in FIG. 2B, which focuses on an enlarged view of area 114. Perforations 113 are disposed through planar portion 111 in a staggered manner and have a generally circular shape. These perforations may be any size, e.g., exhibiting less than 3 mm diameters. In other embodiments perforations 113 may be designed to have other shapes such as an oval, square, rectangle or triangle and portion 111 can include uniformly shaped perforations like are shown in the figures or could include differently sized and/or shaped perforations.

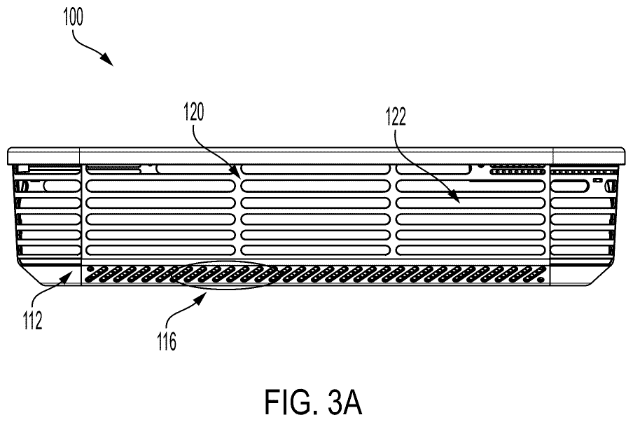

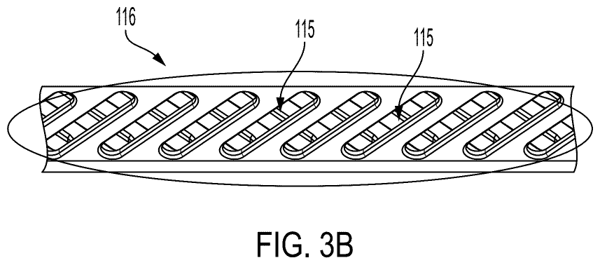

Bottom surface 110 further includes a tapered portion 112 connecting bottom surface 110 to side surfaces 120 of sterilization tray, as best shown in FIGS. 2A-4. Tapered portion 112 of bottom surface 110 includes a plurality of elongated perforations 115. These elongated perforations 115 are shown in more detail in FIG. 3B, which focuses on an enlarged view of area 116. Elongated perforations 115 are situated in a substantially oblong fashion and are spaced apart from one another in a linear row extending along a lateral width of sterilization tray 100. Perforations 15 are shown recessed from a surface of tapered portion, and in fact are included within separately formed slots. In other embodiments, perforations 115 can be differently sized and/or shaped as can tapered portion 112. For instance, it is contemplated that tapered portion 112 could be rounded and perforations 115 could be circular, as opposed to being elongated.

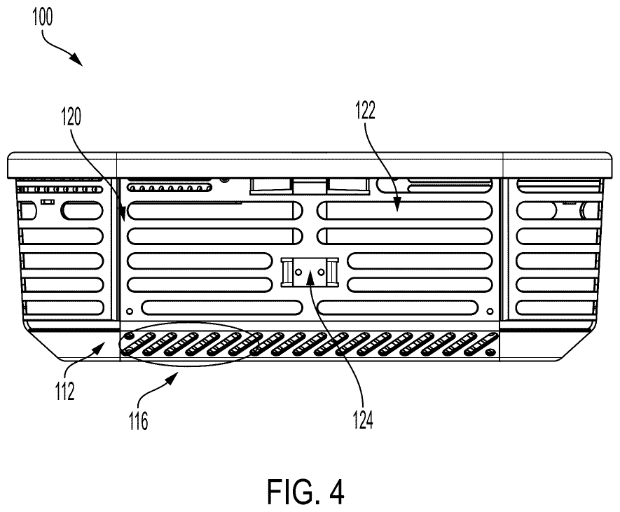

Side surfaces 120 of sterilization tray 100 are best shown in FIGS. 3A-4. As shown, these surfaces connect with tapered portion 112 and each other to define the aforementioned inner space of tray 100. Side surfaces 120 include a plurality of slots 122 extending through each respective side surface 120 into the inner space of the sterilization tray 100. Slots 122 have a substantially oblong shape and extend along the length of each side surface 120 in spaced apart rows and columns. Slots 122 proximal to bottom surface 110 have a smaller width compared to those slots more proximal to top surface 130. In other words, a respective width of each respective slot 122 increases in a direction from bottom surface 110 to top surface 130 along each respective side surface 120. As discussed further below, the different width slots are included in order to maximize total surface area while still preventing components of the medical device from being able to extend through slots 122 and outside of sterilization tray 100.

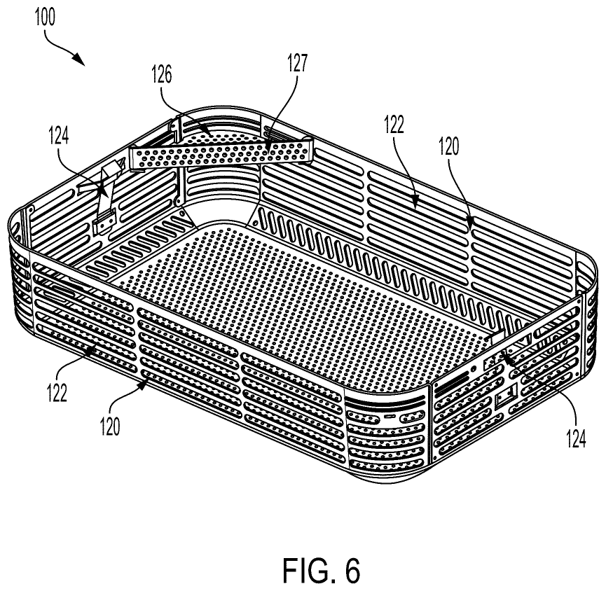

Because of the rectangular nature of tray 100, two longer and two shorter side surfaces 120 are included. The two shorter side surfaces 120 include latches or flared out bosses 124 extending from an inner surface of the side surface 120. Bosses 124 are disposed on side surfaces 120 opposite from one another to releasably engage (as discussed below). Additionally, as best shown in FIG. 6, an internal corner of two adjacent side surfaces 120 of sterilization tray 100 includes an accessories shelf or holder 126 for a chemical indicator (not shown). The chemical indicator may be used for determining whether a sufficient amount of sterilant had entered the inner space of the sterilization tray 100 for a sufficient amount of time. In particular, the chemical indicator positioned within sterilization tray 100 may be compared to a chemical indicator positioned outside sterilization tray 100 during the sterilization process. This is discussed more fully below. Additionally, holder 126 may also be used to include additional components of the medical device, such as buttons, valves, and adapters of an endoscope that are better suited for placement in the holder than in other portions of tray 100. As shown, shelf or holder 126 is connected to two sides of the plurality of sides and includes a security wall 127. This construct aid in receiving and retaining an indicator or other accessory. As shown, holder 126 and wall 127 each include perforations or holes for permitting fluid flow to anything contained within the holder. In other embodiments, the holder may be connected to three sides of the try and could include other sized and/or shaped openings.

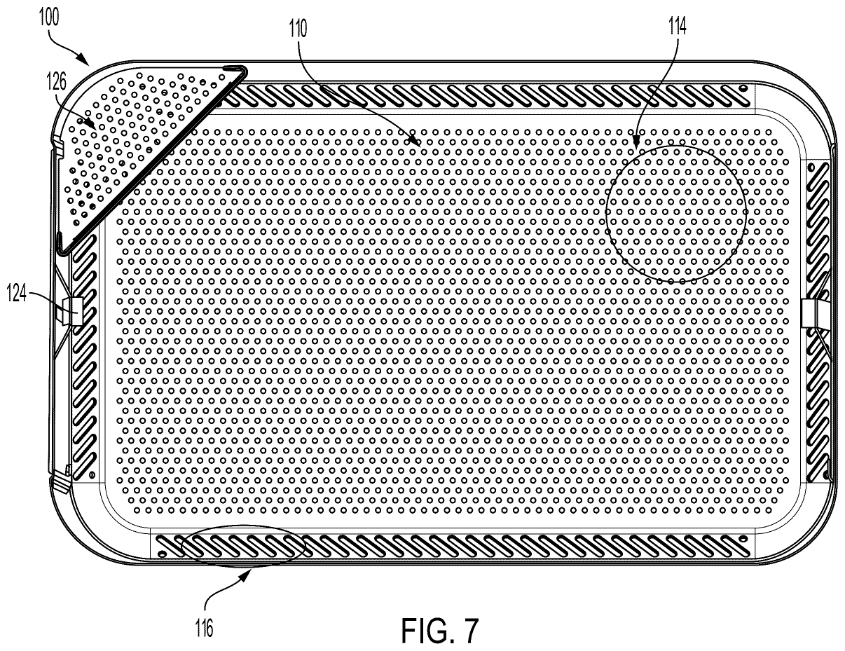

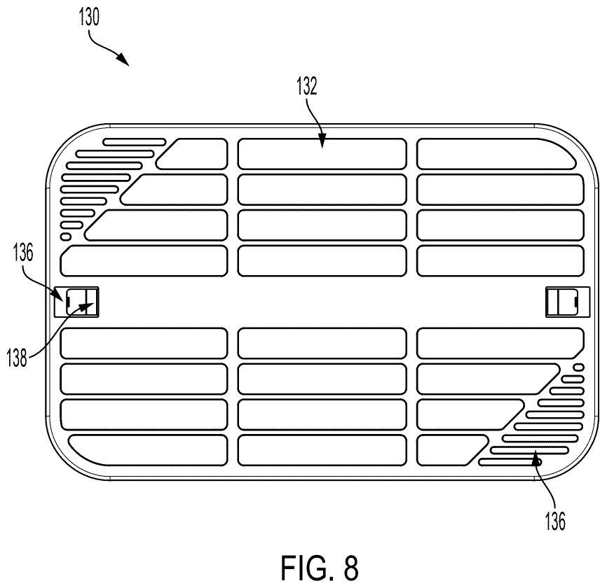

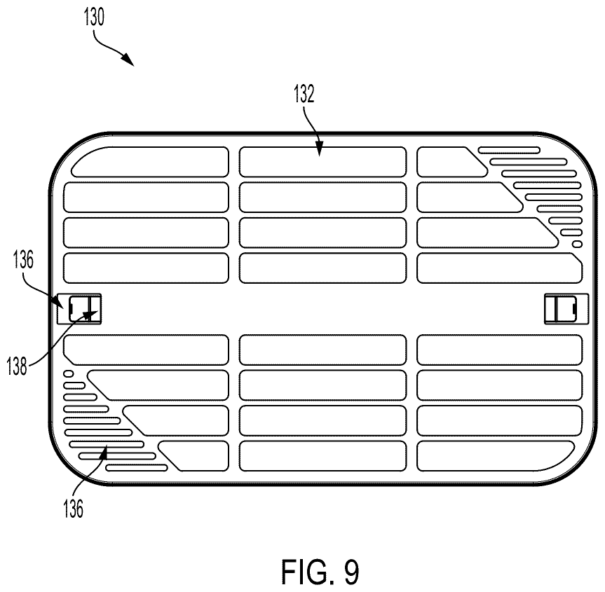

Cover or lid 130 of sterilization tray 100 is best shown in FIGS. 1, 5, 8 and 9. Cover 130 includes a plurality of openings 132 extending through a surface of the cover into the inner space of the sterilization tray 100. While the majority of openings 132 have a substantially rectangular shape and extend laterally along the width of side surfaces 120 in spaced apart rows and columns, certain of the openings are sized and shaped in a particular fashion to permit the inclusion of other structures in the cover. For instance, cover 130 further includes a plurality of corner openings 134 also extending through a surface of cover 130 into the inner space of the sterilization tray 100. Cover 130 also includes openings 136 and buttons 138 for affixing the cover to the remainder of tray 100. In particular, bosses 124 are designed to pass through openings 136 and into engagement with buttons 138. Actuation of the buttons thereafter permits disengagement with the bosses and permits removability of the cover. Depending upon a given situation, sterilization tray 100 may be used in a sterilization process, as discussed further below, without having cover 130 attached or used (i.e., in the manner shown in FIGS. 6-7).

Sterilization tray 100 is designed to have a total surface area along an exterior of the tray that achieves maximum vaporization of residual liquid while still promoting air flow and sterilant diffusion therethrough. Specifically, perforations 113, 115 formed through bottom surface 110, slots 122 formed through side surfaces 120, and openings 132 formed through cover 130 all combine to create a porosity of the total surface area of sterilization tray 100. Sterilization tray 100 may have a porosity ranging from 35% to 45% of the total surface of the exterior, with a preferred porosity of 40-42%. This porosity has been found to maximize the amount of vaporization of residual liquid while maintaining a sufficient level of sterilant diffusion through the tray. The design of tray 100 includes a plurality of specifically sized, shaped and specifically oriented openings that extend through the tray in order to assist with the drying and sterilization process. In particular, the porosity is greater than a minimum area ratio required to achieve efficacy of a sterilization cycle for gastroinstestinal endoscopes or the like. As shown, the openings formed through cover 130 form an open area that is about 200% of that provided by the perforations of the remainder of tray 100. The open area ratio of the remainder of the tray is less than 20% to prevent residual liquid in the tray from freezing, including on the sterilization wrap.

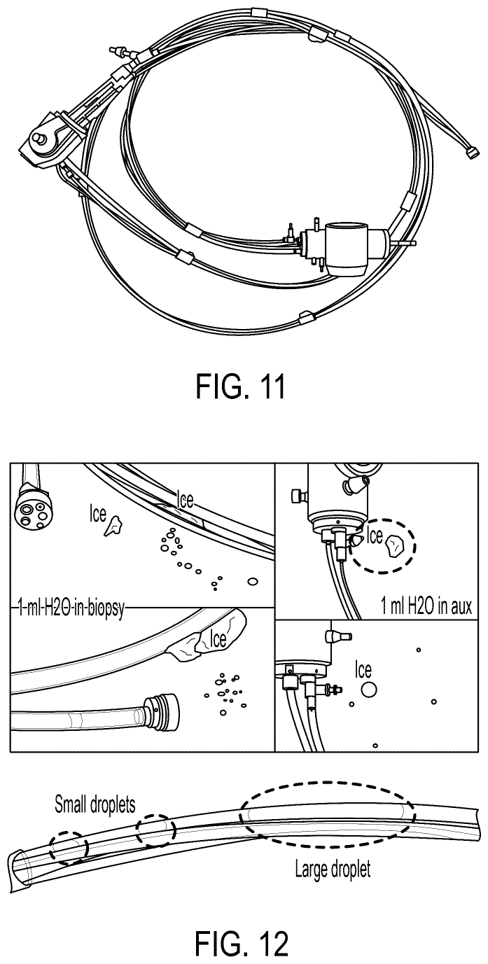

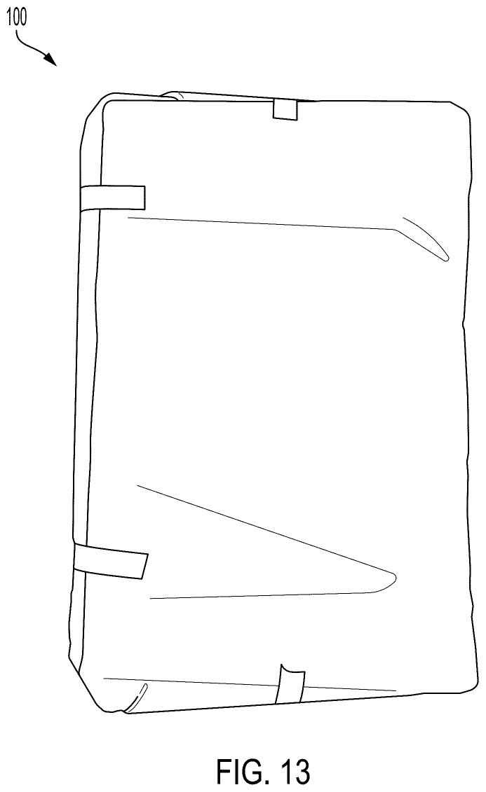

In use, sterilization tray 100 enhances drying and removal of residual liquid (i.e., water or alcohol) of the medical device (for instance the devices shown in FIGS. 10-12) prior to commencing the sterilization process within the sterilization chamber. First, a medical device is positioned within the sterilization tray 100 (see e.g., FIG. 10). After positioning the medical device within sterilization tray 100, sterilization tray 100 may be wrapped within a sterilization wrap to from a sterilization pack or load, as best shown in FIG. 13. The wrap, which may be comprised of a material such as polypropylene, acts as a barrier to microorganisms, but it permits passage of a sterilant therethrough. Wrapping of sterilization tray 100 is not necessary in all embodiments.

Next, with the sterilization tray 100 positioned within the sterilization chamber, the sterilization chamber 100 is heated to vaporize residual liquid from the medical device, sterilization tray, or elsewhere within the sterilization chamber. During this load conditioning process, sterilization tray 100 is heated to assist with vaporizing the residual liquid. As the sterilization chamber is evacuated under pressure, residual liquid will collect within the plurality of perforations 113 of the sterilization tray 100 and will vaporize when contacting the heated bottom surface 110 of sterilization tray 100.

The atmosphere within the sterilization chamber, which may include vapor, is then evacuated to remove residual liquid prior to commencement of the sterilization process. In addition to the above steps, air within the vacuum chamber may be excited to form an air plasma, which may further aid in vaporizing residual liquid for removal from the chamber. The amount of residual liquid removed during the load conditioning process may vary depending upon a weight, materials, total surface area, or other design considerations of the sterilization tray 100. Additionally, the amount of residual liquid removed may depend upon the positioning of the sterilization tray 100 within the sterilization chamber, initial concentrations of residual water or alcohol within the medical device, sterilization tray 100, and variables such as time, temperature, and pressure of the sterilization cycle performed on the sterilization tray 100. The features of the particular sterilization tray 100 shown and discussed herein permits the removal of at least 10-15 milliliters of residual liquid during the load conditioning process and prior to commencement of the sterilization process, including at low pressures (e.g., below 4.6 Torr).

In particular, the plurality of perforations 113 of the sterilization tray 100 are designed to maximize residual liquid to be vaporized during the load conditioning process while also providing the benefit of allowing vaporized sterilant to penetrate bottom surface 110 to contact the medical device during the later performed sterilization process. Specifically, the sterilization tray 100 is designed to minimize the radius of each perforation 113 of the plurality of perforations to maximize the surface area of planar portion 111 of bottom surface 110. The design of perforations 113 and bottom surface 110 in this manner increases the rate of energy transfer between sterilization tray 100 in contact with residual liquid removed from the medical device and collected within each perforation 113 during the load condition process. Thus, minimizing the size of each perforation 113 to maximize the surface area and mass of the planar portion 111 of bottom surface 110 increases the rate of vaporization of residual liquid during the load conditioning process. Further, the size of each slot 122 and the size of each opening 132 are designed to maximum sterilant diffusion through the sterilization tray 100.

Tray 100 includes an overall outer surface area. Perforations 113 define first surface area, slots 122 define a second surface area and openings 132 define a third surface area. The first surface is less than the second surface area and the second surface area is less than the third surface area. These features result in maximal vaporization of residual liquid, while also maximizing sterilant diffusion through the tray. This precludes both issues and delays resulting from residual liquid not being removed from the medical device and a reduction in the overall time required to sterilize the load.

Additionally, the design of the sterilization tray 100 may prevent residual liquid from freezing and rather results in vaporization of that liquid. During the load conditioning process, the sterilization chamber may be brought to a pressure and temperature condition that freezes liquids, such as water. Therefore, if residual liquid is not sufficiently vaporized during the load conditioning process, residual liquid may freeze on a surface of the sterilization tray or on any utilized sterilization wrap. This may impede subsequent sterilization processes, including flow of vaporized hydrogen peroxide (“VHP”), which in turn can lead to unwanted and potentially dangerous VHP condensation on the tray and/or wrap. Both retained moisture and freezing can act to degrade sterilization cycle efficacy. In addition, residual hydrogen peroxide on the tray and/or wrap post-cycle poses a safety hazard to sterilization operators and Further, commercially available sterilization trays may be comprised of a plastic material rather than a metal, residual liquid freezing on a surface of the sterilization tray is even more prevalent. In the above-mentioned sterilization method, sterilization system is configured to not introduce sterilant into the sterilization chamber if enough residual liquid is detected after the load commencing process. The sterilization process will then abort, and the medical device, sterilization tray and sterilization chamber will need to be dried in order to continue with the sterilization process, which increases the overall time and effort to sterilize the medical device.

Further, the design of sterilization tray 100 provides the additional benefit of being able to accommodate larger medical devices compared to that of commercially available sterilization trays. For instance, commercially available sterilization trays are unable to accommodate GI endoscopes and other similar larger medical devices. Sterilization tray 100 provides a larger inner space when compared to commercially available sterilization trays to accommodate these larger medical devices. For instance, the inner space is configured to accommodate a flexible gastrointestinal endoscope having a bending radius for an insertion tube grater than 125 mm.

Furthermore, sterilization tray 100 is designed from materials compatible with the load conditioning and sterilization process discussed above. Sterilization tray 100 may be comprised of aluminum, stainless steel, other commonly used metal alloys, or any other materials suitable to withstand temperatures, pressures, and sterilants (or other chemicals) used during either process. Additionally, sterilization tray 100 is manufactured to accommodate medical devices, which may include a design that is specific or custom to the medical device to be sterilized. For example, bottom surface 110 of sterilization tray 100 may be shaped or included undulations or printed templates to match the contours for medical device to be sterilized. Moreover, while shown largely rectangular, trays according to the present invention may exhibit various shapes.

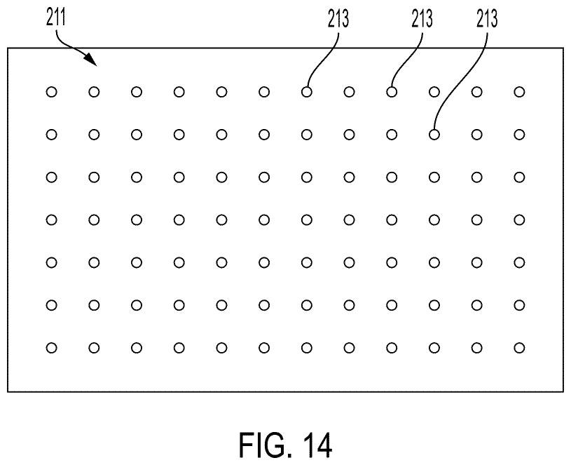

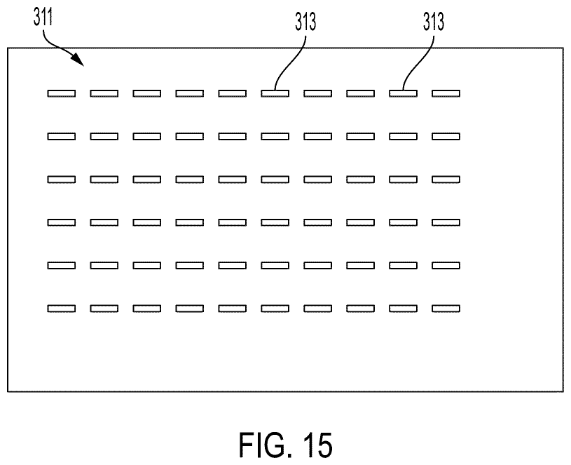

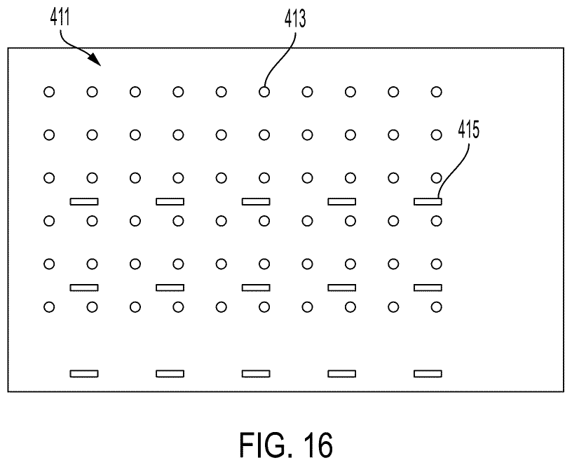

FIGS. 14-16 depict alternative configurations for a bottom surface of the tray. In particular, FIG. 14 depicts a planar portion 211 including uniformly spaced perforations 213. As opposed to the offset nature of perforations 113, perforations 213 are uniformly spaced in a linear column/row fashion. FIG. 15 depicts a planar portion 311 including uniformly spaced perforations 313 in the form of elongate slots. Although also shown in a linear column/row fashion (like perforations 213), it is to be understood that perforations 313 can be arranged in other manners. It is also to be understood that since perforations 313 are each of a larger surface area than perforations 113 and 213, less of them need to be included in the bottom surface of the tray. As shown, perforations 213 include widths of less than 2 mm, although other sizes and shapes (e.g., curved slots) are contemplated. Indeed, the aforementioned prevention of liquid leakage and freezing can be achieved by include less perforations 313 or by sizing them to be smaller elongate openings. FIG. 16 depicts a planar portion 411 including both circular perforations 413 and elongate openings 415. Again, these can be arranged in different manners from that specifically shown in the drawings.

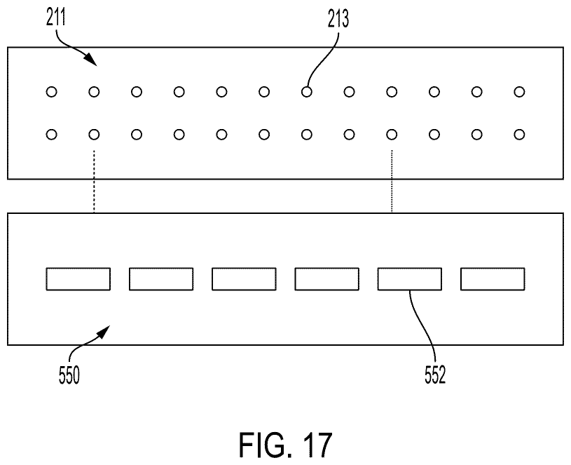

FIG. 17 schematically depicts the inclusion of a baffle 550 that includes elongate openings 552. This baffle can be positioned directly beneath the base of the tray in order to block direct vertical pathways. This can act to prevent residual liquid from dripping straight through the tray. Instead, droplets fall onto the baffle surface, whereby they are more readily permitted to evaporate. Baffle 550 may be positioned such that a defined gap exists between it and the base of the tray (shown as planar portion 311 in FIG. 17) that permits an unobstructed vapor diffusion. Of course, other baffle designs are contemplated, including baffles that have differently shaped openings than those shown in FIG. 17. It is also contemplated to position a baffle with respect to a tray in any known manner, including via a mechanical interconnection or including a specific structure facilitating such positioning with a sterilizer.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A tray for holding medical devices, comprising:
a base having a base surface and a base configuration of openings on the base surface, the base configuration of openings being configured to facilitate diffusion of a sterilant through the base surface into the tray and to prevent residual liquid in the tray from freezing on a sterilization wrap surrounding the tray.

2. The tray of claim 1, further comprising:
a plurality of sides extending from the base, wherein at least one side includes a wall and a side configuration of openings on the wall, the side configuration of openings being configured to facilitate sterilant communication in and out of the tray and to enable vaporization of residual liquid through the side.

3. The tray of claim 2, further comprising:
a lid configured to releasably engage the plurality of sides, the lid having a lid configuration of openings configured to facilitate sterilant communication in and out of the tray and vaporization of residual liquid through the lid,
wherein an open area ratio of the lid is at least 200% of an open area ratio of the base.

4. The tray of claim 3, further comprising:
an accessories shelf having a security wall, the shelf connected to two sides of the plurality of sides, wherein the accessories shelf is configured to receive a sterilization indicator and retain the sterilization indicator based in part on the security wall during and after a sterilization process.

5. The tray of claim 4, wherein the base configuration of openings, the side configuration of openings, and the lid configuration of openings are collectively configured to form a tray open area ratio, the tray open area ratio being greater than a minimum open area ratio required to achieve efficacy of a sterilization cycle for gastrointestinal endoscopes.

6. The tray of claim 5, wherein the minimum open area ratio is 40%.

7. The tray of claim 1, wherein the base configuration of openings has an open area ratio of less than 20% to prevent the residual liquid in the tray from freezing on the sterilization wrap.

8. The tray of claim 1, wherein the base surface comprises at least one metal alloy, and the base surface and the base configuration of openings are configured to:
(1) facilitate a load conditioning process of a sterilization cycle to remove up to 15 milliliters of residual liquid from the tray; or
(2) prevent the residual liquid from freezing on the sterilization wrap at pressures below 4.6 Torr in a vacuum chamber of the sterilizer.

9. The tray of claim 1, wherein the base configuration of openings comprises a plurality of perforations or a plurality of slots, wherein the perforations or slots collectively form an open area ratio of less than 20%.

10. The tray of claim 1, wherein the base surface is a top surface, and the base further comprises a flat bottom surface configured to contact the sterilization wrap to facilitate diffusion of the sterilant through the base configuration of openings into the tray.

11. The tray of claim 1, wherein the base surface has a size configured to accommodate a flexible gastrointestinal endoscope having a bending radius for an insertion tube greater than 125 mm.

12. A method for making a sterilization tray, comprising:
forming a base configuration of openings in a thermally conductive base of the sterilization tray, the base configuration of openings being configured to prevent residual liquid in the tray from freezing on a thermally nonconductive wrap covering the tray during a sterilization process; and
forming a lid configuration of openings in a lid of the sterilization tray, wherein the lid configuration of openings and the base configuration of openings provide an open area ratio of the lid that is at least twice that of the base.

13. The method of claim 12, wherein the base configuration of openings comprises a plurality of perforations or a plurality of slots, wherein the perforations or slots collectively form an open area ratio of less than 15%.

14. The method of claim 12, wherein the base comprises at least one metal alloy, and the base and the base configuration of openings function in synergy to enable a load conditioning process of a sterilization cycle to remove up to 10 milliliters of residual liquid from the tray.

15. A tray for holding medical devices, comprising:
means for preventing residual liquid in the tray from freezing on a sterilization wrap surrounding the tray during a sterilization process; and
means for evacuating the residual liquid from the tray.

16. The tray of claim 15, further comprising:
means for facilitating passage of vaporized sterilant or moisture through a base, a side, or a lid of the tray.
